(19) United States
(12) Patent Application Publication
(10) Pub. No.: US 2007/0128293 A1
(43) Pub. Date: Jun. 7, 2007

(54) Extrapallial fluid of molluscs, methods of obtaining same, formulation and use thereof

(75) Inventors: Evelyne Lopez, Paris (FR); Xavier Bourrat, Bordeaux (FR); Marthe Rousseau, Brest (FR)

Correspondence Address: NIXON & VANDERHYE, PC, 901 NORTH GLEBE ROAD, 11TH FLOOR, ARLINGTON, VA, 22203, US

(73) Assignee: SOCIETE D'INNOVATION ET DE RECHERCHE APPLIQUEE SA (SIERA SA), PARIS (FR)

(21) Appl. No.: 10/587,968

(22) PCT Filed: Feb. 14, 2005
(86) PCT No.: PCT/FR05/00344

§ 371 Date: Aug. 3, 2006

(30) Foreign Application Priority Data
Feb. 13, 2004 (FR) 0401474

(51) Int. Cl. A61K 35/56; A22C 29/04

(52) U.S. Cl. 424/547; 452/12

(57) ABSTRACT

The invention relates to a fluid preparation method comprising the recovery of the fluid that is located between the shell and the body of a mollusc. The invention also relates to the liquid obtained using said method, and to the use thereof in the pharmaceutical field, in particular in order to improve cutaneous healing, to regenerate tissues or to promote osteogenesis or bone mineralization. The invention further relates to compositions, particularly pharmaceutical compositions, medical devices or food supplements containing same.

The invention relates to a method of obtaining a fluid comprising extracting from a mollusc the fluid that is located between the shell and the body of the mollusc. The invention also relates to the liquid obtained using said method, and to the use thereof in the pharmaceutical field, and in particular in order to improve cutaneous healing, to regenerate tissues or to promote osteogenesis or bone mineralization. The invention further relates to compositions, particularly pharmaceutical compositions, medical devices or food supplements containing same.

In many cases of degeneration of mineralized tissues, tissue repair remains a complex problem which has yet to be resolved, in particular due to the difficulty of employing substitute materials inducing regeneration. Among such substitute materials inducing regeneration, mother of pearl has already been shown to have biocompatibility and osteogenic and osteoinduction capacity with respect to human osteoblasts [Lopez et al., Tissue & Cell, 1992: 667-679; Lamghari et al., J. Bone Miner. Res. 2001, 16: 2232-2237].

In this context, mother of pearl has made it possible to offer effective solutions in specific cases. For instance, implants made from mother of pearl have been proposed for endosseous repair. Mother of pearl powder has also been proposed as filler material where the biodissolution properties thereof are manifested: a gradual replacement by newly formed compact and spongy bone is observed, according to the nature of the recipient bone.

Unfortunately, the use of mother of pearl as biomaterial is not always easy or possible in many cases, for example in large local repair of degenerated tissue or loss of substance.

Up to now, then, no biomaterial exists which is easy to formulate and which enables the regeneration of biological or bone tissues in an entirely satisfactory manner.

In this respect, the applicant has developed a method of obtaining the extrapallial fluid located between the shell and the body of a mollusc by extraction of said fluid (or liquid). The applicant has shown that the fluid so obtained can conserve its self-assembly properties and that the latter can be used, even in a context external to the animal. Furthermore, said fluid is of major interest from an industrial standpoint by virtue of its physical nature, since it can exist in many forms, such as for example in the form of a liquid, film, coating, fibers or else in the form of a porous material. Thus, it can be easily manipulated and employed in different forms, all while conserving its biological properties.

The invention is therefore directed at a method of obtaining an extrapallial fluid comprising recovering the extrapallial fluid that is located between the inside of the shell and the body of a mollusc, in particular the mantle of said mollusc.

The invention also has as object the fluid which can be obtained by said method and the use thereof in various fields. The fluid obtained is thus pure and taken in its entirety, in particular it is not modified and not transformed, following recovery, by a process modifying its intrinsic qualities. It can subsequently be formulated, in particular for use in a pharmaceutical composition, medical device or food supplements.

In particular, the pharmaceutical composition according to the invention is intended to improve the phenomena of healing, to regenerate biological or bone tissues, to promote osteogenesis or bone mineralization.

In the animal, said fluid participates in situ in the mineralization of the exoskeleton. Some see an overall unity in the mineralization processes found in nature, from the mollusc exoskeleton to the endoskeleton of higher mammals. Therefore, the range of application of said bioproducts is potentially quite vast, in fields involving the skeleton, bone and cartilage, teeth, or any other tissue, in particular the skin and its appendages.

According to the invention, the fluid preparation method therefore comprises a step of recovering the extrapallial fluid located between the shell and the body of a mollusc, in particular the mantle of said mollusc, said fluid having been extracted from a mollusc.

The fluid recovered in this way corresponds to the natural secretions of the mollusc which are found at the external surface of the mollusc mantle near the shell. In a surprising manner, the applicant has found that said liquid, even after it is extracted and therefore outside its natural context, could be used for its regenerating and mineralizing properties, and thereby promote the formation of the shell (organomineral material) of a mollusc, and more broadly substitute for, or regenerate, the secretions of any other tissue known to form an extracellular matrix. Hence the fluid has the advantage of regenerating biological or bone tissues, in particular by stimulating the activity of the cells which form said tissues or by supplying the building blocks useful for said regeneration, and of promoting osteogenesis or bone mineralization.

Tissue regeneration is understood to mean the reconstruction and renewal of any tissue which has been damaged or altered (or injured tissue). Mineralization (mineralizer or mineralizing) refers to the action (capacity) which consists in supplying mineral elements or promoting the fixation of mineral elements on the organic material. Mineral species include crystals, atoms, ions, molecules containing “mineral” elements and/or trace elements, as opposed to organic elements. Thus, the regeneration of a biological or bone tissue generally consists in reconstructing or renewing all or part of the altered tissue, in particular with the aid of molecules having a specific structure, “signal” molecules and organic and/or mineral nutrients present in the fluid obtained according to the invention. Furthermore, no intolerance was observed when the fluid so extracted and/or subsequently modified was applied on or in a mammal.

The step of collecting or extracting the fluid is generally carried out in the following manner:

The mollusc from which the fluid is extracted is generally a mono- or bi-valve mollusc, a mother of pearl mollusc or not, advantageously mother of pearl. In particular, the molluscs are oysters and more specifically oysters of the genus Pinctada and more particularly of the genus Pinctada species margaritifera or maxima. The oysters can also be selected in the group consisting of the genus Ostrea, in particular the species edulis, or else the genus Crassostrea, in particular the species gigas. The molluscs can also be cephalopods, such as for example the nautilus, or else gastropods, such as for example the winkle.

The mollusc which is used can be from a bottom or off-bottom culture. The mollusc can be sacrificed before, during or after the inventive method. It can be used dead or alive. For example the mollusc can be sacrificed prior to the inventive method, taking care to at least partially (preferably totally) conserve the fluid to be extracted in the shell.

Collection of extrapallial fluid can be carried out on batches of molluscs devoted solely to this use, without this harming the integrity thereof. The molluscs can then be used at a later time, as they remain intact and alive.

More specifically, the extraction process or step is a noninvasive method. In fact, the fluid located between the inside of the shell and the body of the mollusc, in particular the mantle of said mollusc, corresponds to the natural secretions of the mollusc which are found at the outer surface of the mollusc mantle near the shell and which are therefore located outside the mollusc body itself.

In order to access the fluid to be extracted, the mantle of the mollusc is preferably detached in whole or in part. In the case of a bivalve mollusc, at least one of the two valves is lifted so as to be able to access the body of the mollusc, prior to extraction.

The detachment (or separation) of the fluid from the shell can be carried out manually, generally with the aid of a suitable tool (such as for example a scraper or flexible spatula). According to another method, optionally in addition to said manual detachment, the fluid is detached from the shell by chemical means. For instance, an enzymatic reaction, advantageously non-denaturing, and generally at room temperature, can also be employed so as to more finely separate the fluid from the shell. Preferably, said enzymatic reaction is carried out in the presence of at least one protease, such as trypsin for example. The reaction can then be stopped by a protease inhibitor or by simple dilution.

The fluid is then collected at the surface of the shell with the aid of a suitable instrument, in particular with the aid of a syringe (in particular equipped with a needle).

The collected fluid is advantageously sterilized or decontaminated by any known method, in particular by filtration, for example on a Millipore® filter (example: 0.20 μm). The fluid can also be dehydrated, in particular by freeze-drying. The fluid can also be frozen, preferably immediately after recovery or after filtration.

According to a variant of the method, the collection of fluid can be carried out on living molluscs, for example in the case of large molluscs, such as the mother of pearl bivalves of the Pinctada family.

Preferably, said variant consists in keeping the shell of the living bivalve open with the aid of an instrument, such as a wedge and/or plier (or retractor). The instrument is preferably selected so that it does not injure the animal, in particular it is selected so that it does not irreversibly alter the metabolism thereof. The mantle is preferably lifted in whole or in part in order to separate it from at least one of the shells.

The fluid is then collected at the surface of the shell with the aid of a suitable instrument, such as in particular a scraper or flexible spatula. Advantageously, a scraper (plastic) is used to collect the fluid located between the mantle and the shell. Alternatively, the fluid is collected with the aid of a syringe generally equipped with a needle, preferably having a caliber comprised between 1 mm and 5 mm. Of course, any other material adapted to the collection of the fluid can be envisioned.

The method of obtaining the fluid according to the invention can additionally comprise subsequent non-denaturing steps on the fluid so recovered, such as steps of filtration, decontamination, sterilization, freezing/thawing, modifications of viscosity, and/or various transformations.

The fluid obtained by the aforementioned method can be used as is, in pure state (taken in its entirety), in particular it is not modified and not transformed, following recovery, by a process which alters its intrinsic qualities (denaturing steps), in particular by a process of demineralization.

The fluid which is obtained can then be formulated according to its subsequent use. For instance, the subsequent use of the fluid may require a suitable mode of preparation. Experiments conducted with the recovered fluid show that it is very easy to manipulate and that it adapts very easily to all sorts of constraints related to the use thereof. For instance it can undergo several freeze-thaw cycles without this affecting its biological properties. The same is true for freeze-drying. Of course, the person of the art will take care not to modify the intrinsic qualities of the extracted fluid and will therefore avoid subjecting said fluid to any operation which detracts from its value, such as a demineralization step.

It is also possible to change the viscosity of the fluid so obtained by modifying the water content thereof. A gelling agent or any other substance facilitating the subsequent use of the fluid can be added, in order to make it more or less liquid (viscous).

For example, the fluid can be transformed into a film or a coating. Any known method whereby a liquid can be transformed into a film or a coating can be employed. For example the fluid can be spread on a smooth or porous surface or any other support and form a film (filmogenic character of the fluid obtained). The film or coating thus formed retains the regenerating and healing character of the fluid. It can thus form a biological organic surface film, with mineralizing power. The thickness of the film can vary over a wide range according to its final use. For example a thick film can be obtained with the triple property : (i) impermeability, (ii) self-supported structure, (iii) intrinsic supply of minerals, all while displaying the regenerating and healing character. Said material, composed of a close association between organic material and mineral material, forms a film on any surface and crystallizes in a polymorphous manner according to the state and texture of the surface. The film so obtained can be used on its support or removed from the support for a later use.

The fluid can also be subsequently used in the form of fibers. Any known method whereby a liquid can be put into fiber form can be employed. In fact, said fluid has the advantage of being spinnable over a wide range of viscosities. In particular, a very simple drawing method, in particular with the aid of tweezers, makes it possible to draw fibers from the fluid. In this way it can be used in situ (locally) or in another way, as porous filler material, invasive, in order to fill a given volume as needed. In particular, the invasive character of said material allows biological materials, such as cells, to penetrate therein and to exert their effects therein in association with the properties of the transformed fluid. The same material can be used to bridge two distant edges of tissue. The same principle can be applied on very large volumes, in particular by coating with fibers having another chamical nature (for example carbon fibers). The fibers so coated have a surface coat comprising the components of the fluid according to the invention and can be used for example to provide a healing interface.

The fluid can thus exist in different forms, including liquid, films, coatings and fibers. Depending on the desired transformation, it can also take the form of a porous material, in particular by using a porous support during its transformation. The porous material can be a film, a coating or exist in any other form, such as a spherical form.

The inventive fluid generally comprises a mixture of the constituent elements of living matter, in particular a mixture of proteins, proteoglycans, polysaccharides, lipids and mineral salts, which is more particularly the case for a mother of pearl mollusc, advantageously an oyster of the genus Pinctada (generally harvested in its natural biotope). The nature and quantity of these compounds are subject to large variations, particularly as a function of the mollusc, the biotope of same, the season of harvest and the harvesting conditions.

To give a rough idea, said elements were analyzed in a given fluid (adult oyster of the genus Pinctada harvested in its natural biotope). The composition of said fluid preferably comprises at least the following compounds : proteins at an average concentration of 0.5 mg/ml, proteoglycans at an average concentration of 3 mg/ml, polysaccharides at an average concentration of 1.5 mg/ml, lipids at an average concentration of 1 mg/ml, and mineral salts at an average concentration of 7.5 mg/ml.

A quantitative analysis was carried out on the trace elements present in a specific fluid (identical to that identified hereinabove) according to the invention, in particular by quantitatively measuring the trace elements present by neutron activation analysis (NAA) and inductively coupled plasma mass spectrometry (ICP-MS). The composition of said fluid (obtained directly : without dehydration and subsequent modification) preferably comprises trace elements selected from: S, Mg, La, Zn, Br, Ce, Fe, Mn, Cl, Cu, K, Sr, Na and Ca. Preferably, the respective quantities of said trace elements are as follows: 0.02; 2; 0.28; 0.4; 1.78; 5.5; 13.6; 50; 296; 143; 582; 1000; 5420 (expressed in μg/g) and 38.8 (expressed in μg/100 g).

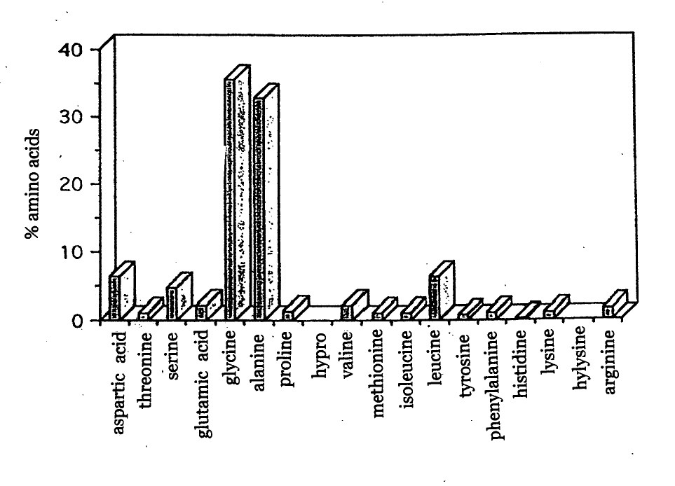

A quantitative analysis of the amino acids present in free or bound form in an inventive fluid was carried out, in particular by quantitatively measuring the amino acids present with a Beckmann 6300 amino acid analyzer using the classical ninhydrin method. The result of this analysis is given in FIG. 1 (% of amino acids by mass relative to total amino acid mass).

The invention also has as object the fluid which can be obtained by the method such as described hereinabove. The liquid (or fluid) can not only be the liquid obtained directly after the recovery step described hereinabove, but also that obtained after subsequent non-denaturing steps, such as steps of filtration, decontamination, sterilization, freezing/thawing, modifications of viscosity, and/or various transformations, such as those noted hereinabove in particular.

More particularly, the inventive fluid is used as a medicament.

The invention also has as object a composition, in particular pharmaceutical, characterized in that it comprises the fluid such as defined hereinabove, in particular in association with a pharmaceutically acceptable excipient.

The fluid used as medicament or in the composition described hereinabove can be that obtained directly after the recovery step described hereinabove, but also that obtained after subsequent steps of filtration, decontamination, sterilization, freezing/thawing, modifications of viscosity, and/or various transformations, such as those noted hereinabove in particular.

The pharmaceutical composition or the medicament according to the invention can be administered by the topical, enteral or parenteral route. Preferably, the pharmaceutical composition is prepared in a form suitable for topical application, that is to say, applied in situ at the site of tissue damage.

By the enteral route, the pharmaceutical composition can be in the form of tablets, capsules, lozenges, syrups, suspensions, solutions, powders, granules, emulsions, microspheres or nanospheres or lipid or polymeric vesicles allowing controlled release. By the parenteral route, the composition can be in the form of solutions or suspensions for infusion or injection.

According to a particular embodiment of the invention, the pharmaceutical composition is intended for topical use on or in damaged tissue. Thus, the pharmaceutically acceptable support is in particular an excipient suited to topical application.

By the topical route, the pharmaceutical composition according to the invention is more particularly intended for the treatment of the aforementioned tissues, in particular for the treatment of the skin and bones of a mammal, particularly human. It can be in the form of salves, creams, milks, ointments, powders, saturated buffers, solutions, gels, sprays, lotions or suspensions. It can also be in the form of microspheres or nanospheres or lipid or polymeric vesicles or polymer patches or hydrogels allowing controlled release. Said topical composition can be in anhydrous form, in aqueous form or in the form of an emulsion (water/oil, oil/water or multiple emulsion).

The inventive liquid is used, preferably by the topical route, at a concentration generally comprised between 0.02% and 20% by weight, preferably between 0.25% and 10% by weight, and advantageously between 0.5% and 5%, relative to the total weight of the composition

In particular, the therapeutic composition according to the invention is characterized in that it contains, in combination with inactive excipients, a therapeutically effective amount of the inventive fluid, in particular in order to improve cutaneous healing, to regenerate tissues, or to promote osteogenesis or bone mineralization. The tissues comprise soft or skeletal tissues, including in particular the skeleton, bones, cartilage, teeth, or any other tissue, in particular the skin and its appendages. The inventive composition enables the treatment of bone, cartilage and dental disorders in particular.

The invention is also based on a method for improving cutaneous healing, for regenerating tissues, particularly those mentioned earlier, or for promoting osteogenesis or bone mineralization, comprising administering to subjects requiring such treatment a therapeutically effective amount of the liquid such as defined hereinabove.

In the spirit of the invention, the term “treatment” denotes preventive, curative, palliative treatment as well as management of patients (relieving pain, improving quality of life, slowing the progression of the disorder, trauma or disease), and the like.

Furthermore, the method or the treatment can be carried out in combination with other ingredients or treatments, such as in particular other compounds active in improving skin healing, regenerating tissues, or promoting osteogenesis or bone mineralization.

The pharmaceutical compositions or medicaments according to the invention can also comprise at least one other therapeutically active ingredient, for use that is concurrent, separate or spread out over time.

The pharmaceutical compositions or mediaments according to the invention advantageously comprise one or more vehicles or excipients which are inert, that is to say, inactive and nontoxic. Examples include pharmaceutically compatible saline, physiologic, isotonic, buffered solutions and the like, known to those skilled in the art. The compositions can contain one or more agents or vehicles selected from among dispersives, solubilizers, stabilizers, preservatives, and the like. Agents or vehicles that can be used in the formulations (liquid and/or injectable and/or solid) comprise in particular methylcellulose, hydroxymethylcellulose, carboxymethylcellulose, cyclodextrins, polysorbate 80, mannitol, gelatin, lactose, vegetable or animal oils, acacia and the like. The compositions can be formulated as injectable suspensions, gels, oils, tablets, suppositories, powders, gelatin capsules, capsules, and the like, possibly by means of pharmaceutical forms or devices allowing sustained and/or delayed release. For this type of formulation, at least one agent such as cellulose, carbonates or starches is advantageously used.

It is understood that the aforementioned compositions can also contain pharmacodynamically active additives or a combination of said additives, and in particular: wetting agents; emollients; moisturizing agents, like glycerol, PEG 400 or else urea.

Said compositions can also contain flavor enhancers, preservatives such as the esters of para-hydroxybenzoic acid, stabilizers, moisture-regulating agents, pH-regulating agents, agents that modify osmotic pressure, emulsifiers, antioxidants, such as alpha-tocopherol, butylhydroxyanisole, or butylhydroxytoluene

It is understood that the person of the art will take care to select any component(s) to be added to said compositions in such as way that the advantageous properties intrinsically related to the invention are not at all or not substantially modified by the planned addition

Administration can be carried out by any method known to those skilled in the art, in particular by the oral or topical route or by injection, typically by the intraperitoneal, intracerebral, intrathecal, intravenous, intra-arterial or intramuscular route. Administration by the oral or topical route is preferred. In the case of a long-term and non-topical treatment, the preferred route of administration will be sublingual, oral or transcutaneous.

For injections, the compounds are generally prepared in the form of liquid suspensions, which can be injected through syringes or by infusion, for instance. It is understood that the injection rate and/or injected dose, or generally speaking the dose to be administered, may be adapted by those skilled in the art according to the subject to be treated, the disorder, the mode of administration, etc. It is understood that repeated administrations may be given, possibly in combination with other active ingredients or any pharmaceutically acceptable vehicle (buffers, saline, isotonic solutions, in the presence of stabilizers, etc.).

According to a particular aspect, the invention relates to a device, more specifically adapted to subcutaneous or percutaneous injection, comprising the liquid such as defined hereinabove and a physiologically acceptable excipient or support. In particular, said device can be in the form of syringes or infusions.

According to a variant, the medical device of the invention can be intended for implantation in or on the body of a mammal, the aforementioned liquid located inside a membrane adapted to said use.

According to another particular aspect, the invention relates to a food supplement comprising the liquid such as defined hereinabove. In particular, said food supplement is intended to improve cutaneous healing, regenerate tissues, or promote osteogenesis or mineralization of bone or teeth.

The invention can be used in mammals, particularly in humans.

Other aspects and advantages of the invention will become apparent in the following examples, which are given for purposes of illustration and not by way of limitation.

### EXAMPLES

### Example 1

### Recovery of Fluid from an Animal which has been Sacrificed

The mollusc was sacrificed in such a way that the fluid remained inside the shell. The mantle was carefully detached and an enzymatic reaction was then used to completely separate the fluid from the shell. The reaction was stopped with a protease inhibitor. The fluid was then collected at the surface of the shell. Immediately after collection, the fluid can be frozen. It can be sterilized by filtration on a Millipore 0.20 μm filter and be freeze-dried, all while conserving the properties thereof.

### Example 2

### Recovery of Fluid from a Live Animal

An oyster of the species Pinctada margaritifera was opened with the aid of a 25 retractor according to practices well known to those skilled in the art of oyster culture. Excess water still present was eliminated. The animal was laid horizontally, the mantle was gently separated from the bottom shell with the aid of a tweezers, and the fluid was collected with the aid of a syringe, for example.
For group 1: average size: 77 mm average thickness: 26.12 mm, 200 to 300 μl of fluid were collected.
For group 2: average size: 85.2 mm average thickness: 25.6 mm, 300 to 500 μl of fluid were collected.
For group 3: average size: 104 mm average thickness: 36.9 mm, 500 to 800 μl of fluid were collected.
For group 4: average size: 108.6 mm average thickness: 36 mm, 800 to 1000 μl of fluid were collected.

The fluid was:
filtered
freeze-dried
a few weeks later, the fluid was reconstituted with sterile water
it was spread in the form of a thick film.

### Example 3

### Recovery of Fluid from a Non-Mother of Pearl Mollusc

An oyster of the species Ostrea edulis was sacrificed. The shell was opened by shearing the muscle without separating the valves (for example at an angle of 60° from each other). The oyster was immobilized and the mantle detached. In this way, it was possible to collect the fluid from the first valve. The animal was then turned over to repeat the operation on the other side. In this case, 50 to 100 μl of fluid per valve were obtained.

The fluid collected on a group of about twenty animals was then treated as in example 2.

The fluid was:
filtered
freeze-dried
a few weeks later, the fluid was reconstituted with sterile water
it was spread in the form of a thick film.

It was found that the fluid could be reconstituted even after freeze-drying, that it formed a film on a polypropylene support. Said film solidified by self-assembly and self-healing.

Examination under a light microscope showed that it was composed of an organic fraction and a mineral fraction.

1-27. (canceled)

28. A method of obtaining an extrapallial fluid, wherein it comprises recovering the extrapallial fluid located between the shell and the body of a mollusc, in particular the mantle of said mollusc.

29. The method according to claim 28, wherein the mollusc from which the fluid is recovered is a bivalve mollusc, advantageously mother of pearl.

30. The method according to claim 28, wherein the mollusc from which the fluid is recovered is selected in the group consisting of oysters and more specifically oysters of the genus Pinctada and more particularly the species margaritifera or maxima, of the genus Ostrea, in particular the species edulis, or of the genus Crassostrea, in particular the species gigas.

31. The method according to claim 28, wherein the mollusc from which the fluid is recovered is a cephalopod, such as for example the nautilus, or else a gastropod, such as for example the winkle.

32. The method according to claim 28, wherein, prior to recovery, the mantle of the mollusc is detached in whole or in part.

33. The method according to claim 28, wherein, prior to recovery, when the mollusc is bivalve, at least one of the two valves is lifted so as to be able to access the body of the mollusc.

34. The method according to claim 28, wherein, prior to recovery, the detachment of the extrapallial fluid from the shell is carried out manually, generally with the aid of a suitable tool.

35. The method according to claim 28, wherein the extrapallial fluid is recovered at the surface of the shell with the aid of a suitable instrument, such as in particular a scraper, spatula or syringe.

36. The method according to claim 28, wherein it additionally comprises non-denaturing steps on the fluid so obtained, in particular one or more steps of filtration, decontamination, sterilization, freezing/thawing, modifications of viscosity, and/or various transformations.

37. The method according to claim 28, wherein the fluid which is recovered is sterilized or decontaminated

38. The method according to claim 28, wherein the fluid which is recovered is dehydrated, particularly by freeze-drying.

39. The method according to claim 28, wherein the fluid which is recovered is frozen, preferably immediately after recovery or after filtration.

40. The method according to claim 28, characterized in that the fluid which is recovered is transformed into a film or coating.

41. The method according to claim 28, wherein the fluid which is recovered is transformed into fibers.

42. The method according to claim 28, wherein the fluid which is recovered is transformed into a porous material.

43. A fluid which can be obtained by the method such as defined in claim 28.

44. A pharmaceutical composition, wherein it comprises the fluid defined in claim 43 and a pharmaceutically acceptable excipient.

45. The composition according to claim 44, intended to improve cutaneous healing, to regenerate soft or skeletal tissues, or to promote osteogenesis or bone mineralization.

46. The composition according to claim 44, for the treatment of bone, cartilage or dental disorders.

47. The composition according to claim 44, wherein the pharmaceutically acceptable support is an excipient suited to topical, enteral or parenteral administration.

48. The composition according to claim 44, wherein the pharmaceutically acceptable support is an excipient suited to topical application.

49. The composition according to claim 44, wherein it additionally comprises at least one other therapeutically active ingredient for a use that is concurrent, separate or spread out over time.

50. The fluid obtainable by the method as defined in claim 28, as a medicament.

51. A device comprising the fluid obtainable by the method as defined in claim 28 and a physiologically acceptable excipient or support.

52. The device according to claim 51, wherein said devise has a form which is adapted to subcutaneous or percutaneous injection, in particular in the form of a syringe or infusion.

53. The device according to claim 51, wherein the fluid is inside a membrane.

54. A food supplement comprising the fluid obtainable by the method as defined in claim 28.
